(19) United States
(12) Patent Application Publication
(10) Pub. No.: US 2024/0301505 A1
(43) Pub. Date: Sep. 12, 2024

(54) URINARY TRACT CANCER TREATMENT GUIDED BY MUTATIONAL LANDSCAPE

(71) Applicant: BROWN UNIVERSITY, Providence, RI (US)

(72) Inventors: Wafik S. El-Deiry, Providence, RI (US); Taylor E. Arnoff, Providence, RI (US)

(21) Appl. No.: 18/578,390

(22) PCT Filed: Jul. 13, 2022
(86) PCT No.: PCT/US2022/036890

§ 371 Date: Jan. 11, 2024

Related U.S. Application Data
(60) Provisional application No. 63/221,025, filed on Jul. 13, 2021.

(51) Int. Cl. C12Q 1/6886; A61K 31/357; A61K 31/513; A61K 31/675; A61K 33/243; A61K 38/14; A61K 45/06; C12Q 1/686

(52) U.S. Cl. CPC C12Q 1/6886; A61K 31/357; A61K 31/513; A61K 31/675; A61K 33/243; A61K 38/14; A61K 45/06; C12Q 1/686; C12Q 2600/106; C12Q 2600/156

(57) ABSTRACT

A method of treating urinary tract cancer in a subject in need thereof is described. The method includes determining if there are loss-of-function mutations in the CDKN1A and RB1 genes in a biological sample from the subject; and treating the subject with a combination of a checkpoint kinase inhibitor and a DNA damaging agent if there are loss-of-function mutations in the CDKN1A and RB1 genes. Alternately, the method includes determining if there is a loss-of-function mutations in a CDKN1A gene in a biological sample from the subject; determining if there is a mutation in a second gene selected from the list consisting of RAB44, TERT, MUC16, HRNR, and FLG; and selecting specific anticancer treatment for the subject based on the identification of a mutation in the second gene.

### CROSS-REFERENCE TO RELATED APPLICATIONS

This application claims priority to U.S. Provisional Application Ser. No. 63/221,025, filed on Jul. 13, 2021, which is hereby incorporated by reference in its entirety.

### BACKGROUND

Urinary tract cancers are among the most common cancers in the United States. Prostate cancer, bladder cancer and kidney cancer are most common in men while leading cancers in women are bladder cancer and kidney cancer. Among urinary tract cancers, the mortality of bladder cancer is the second high and is increasing year by year. Patients with kidney cancer being detected at early stage are of clinical important. For patients at early stage, surgical resection offers the only chance for cure. After surgery, the five-year survival rate is as high as 80%. Chemotherapy, immunotherapy and target therapy are recommended for late-stage kidney cancer patients. Yet the prognosis is poor and the five-year survival rate is only 22%.

In the United States in 2021, there will be an estimated 83,730 new cases of urinary bladder carcinoma and 17,200 deaths. A strong male prevalence is observed, with almost 75% of all cases occurring in men, and tumors most commonly arise in the seventh decade of life. This disease can present as non-muscle invasive bladder cancer (NMIBC), muscle invasive bladder cancer (MIBC), or as a metastatic form, and each has different molecular drivers. Through whole-transcriptome mRNA profiling, bladder cancer was revealed to have one of the highest mutation rates of any cancer sequenced to date, following only lung cancer and melanoma. Lawrence et al., Nature, 499:214-218 (2013).

Bladder carcinoma has a 6%5-year survival-rate for metastatic disease, with poorly understood links between genetic and environmental drivers of disease development, progression, and treatment response. Rhode Island has among the highest annual age-adjusted incidence rate of bladder cancer at 26.0/100,000 compared to 20.0 in the US, with a paucity of known driver genes for targeted therapies or predictive biomarkers.

Next-generation sequencing technologies have helped to elucidate the genomic complexity of bladder carcinomas. Overall, somatic gene alterations are most common in pathways related to p53, the cell cycle, and RAS-PI3K, in addition to epigenetic modifications. Many tumors display missense or truncating mutations in TP53, driving loss-of-function. Additionally, homologous deletions and truncations are common in cell cycle genes, resulting in the inactivation of genes such as CDKN2A, CDKN2B, RB1, and CDKN1A. Gain-of-function mutations are predominantly seen in FGFR3, PIK3CA, ERBB2, and ERBB3 (Zangouei et al., Cancer Cell Int., 18:127 (2020)), promoting tumorigenesis. Moreover, bladder cancer exhibits significant epigenetic dysregulation at the level of DNA methylation (Kandimalla et al., Nat Rev Urol., 10:327-335 (2013)). DNA hypermethylation is linked to the silencing of a number of tumor suppressor genes, including TP53, RB1, CDKN2A, and CDH1, and is associated with more aggressive disease (Robertson et al., Cell, 171:540-556. e5252017 (2017)). Bladder cancer also has a higher mutational load than most cancers in chromatin remodeling genes, such as inactivating mutations in ARID1A, a SWI/SNF chromatin remodeling subunit, and the histone demethylase KDM6A (Tran et al., Nat Rev Cancer, 21:104-121 (2021)). This suggests that loss of epigenetic regulation may also help promote bladder tumorigenesis.

Furthermore, next-generation sequencing has helped to identify specific molecular subgroups. NMIBC shows a predominance of deletions in CDKN2A, and mutations in FGFR3, PI3K, and TERT have been identified as early drivers of malignancy. Among all cancer types, MIBC has the highest enrichment of APOBEC-specific mutations, with most APOBEC-specific mutations found in the gene promoter of TERT (Roberts et al., Nat Genet., 45:970-976 (2013)). Tumors with APOBEC enrichment, termed APOBEC-high, are more likely to have mutations in DNA damage response genes (TP53, ATR, BRCA2) and chromatin regulatory genes (ARID1A, MLL, MLL3) (Glaser et al., Oncotarget. 9:4537-4548 (2017)). By contrast, APOBEC-low tumors are more likely to have mutations in FGR3 and KRAS. Yet, despite continuing efforts to identify genetic drivers of disease, precision therapies for bladder cancer remain scarce.

Mainstay treatments for bladder cancer currently depend on whether tumors present with muscle invasiveness. NMIBC is treated with endoscopic resection and adjuvant immunotherapy with Bacillus Calmette-Guerin (BCG), but patients who fail to respond to BCG subsequently have limited therapeutic options. MIBC, in contrast, is treated with more aggressive therapies, including radical cystectomy, a cisplatin-based combination neoadjuvant chemotherapy regimen, specifically cisplatin-gemcitabine and radiation. Nevertheless, the benefits of chemotherapies are limited to a subset of patients, and the inability to predict responsiveness remains a major challenge.

Our previous work has demonstrated that sensitivity to cisplatin-based chemotherapies is induced by inactivation of CDKN1A, the gene encoding the cyclin dependent kinase inhibitor p21WAF1 (Sikder et al., Mol Cancer Res., 19:403-413 (2021)). Cisplatin induces DNA adducts, which halts cell proliferation and activates the DNA damage response. Cells deficient in p21 are less able to repair cisplatin-induced DNA adducts, resulting in a greater extent of DNA damage after p21 loss. Loss of p21 also prevents CDK activation, driving progression through the cell cycle without efficient repair of DNA damage. This results in procession down an apoptotic pathway and helps to explain sensitization to cisplatin. Therefore, mutation in CDKN1A has the potential to serve as a candidate biomarker to predict chemotherapy responsiveness. Moreover, CDKN1A has been implicated as a prognostic marker in bladder cancer, as lower p21 expression has been associated with advanced pathologic stage, tumor grade, and lower overall survival (Tang et al., Int J Clin Exp Pathol. 8:4999-5007 (2015)). Further characterization of additional genes dysregulated in concordance with CDKN1A is needed to better elucidate the mechanisms driving disease and to enhance options for precision therapies.

In addition to knowledge of particular genes involved in tumorigenesis, it has been demonstrated that exposure to a number of environmental agents and chemicals are closely associated with an increased risk of developing bladder cancer. The most notable risk factor is occupational exposure to aromatic amines, including 2-naphtylamine, 4-aminobiphenyl, and benzidine, and 4,4′-methylenebis(2-chloroaniline); these chemicals are found in the products of chemical, dye, and rubber industries, as well as in fungicides, plastics, metals, and motor vehicle exhaust (Letašiová et al., Environ Health., 11(Suppl 1):S11 (2012)). Moreover, cigarette smoking is a known primary risk factor for bladder cancer, resulting in a threefold higher risk of developing disease in smokers (Zeegers et al., Cancer, 89:630-639 (2000)). Carcinogenesis induced by smoking is attributed to the presence of chemicals in tobacco smoke, particularly 2-naphthylamine and 4-aminobiphenyl. There is also strong evidence that links the development of bladder cancer with exposure to arsenic in drinking water. Despite understanding that these environmental carcinogens contribute to tumorigenesis, further work is needed to elucidate the particular genes affected by these chemicals as well as the mechanisms that drive transformation.

Current efforts focus on further defining the mutational landscape of bladder tumors to enhance molecular characterization as well as to identify actionable subgroups. Importantly, predictors of chemosensitivity are needed to avoid preventable toxicity and the delay of life-saving radical cystectomies in patients who will prove to be resistant (Tse et al., Int J Mol Sci., 20:793 (2019)). Evaluation of DNA and RNA from patients' urine has recently been approved as a diagnostic marker (Santoni et al., Front Oncol., 8:362 (2018)), and new panels include the measurement of gene expression levels, sequence variations, histone modifications, and DNA methylation. Such advancements are continuing to drive the development of precision therapies.

### SUMMARY OF THE INVENTION

Targeted therapies are still lacking for patients with bladder carcinomas, and this potential combination therapy could prove superior to cisplatin therapy alone. There are currently no FDA-approved treatment options for the use of ATR, CHK1, or CHK2 inhibitors for patients with bladder cancer. We have uncovered a subset of patients with bladder cancers that harbor truncating mutations in both p21 and pRb. Such tumors with cell cycle checkpoint defects could be treated with combination therapies with cisplatin and checkpoint kinase inhibitors. Deregulation of G2 checkpoints in tumor cells with G1 cell cycle checkpoints due to gene mutations that are exposed to DNA damaging agents undergo cell death. The proposed precision oncology strategy offers potential benefit to patients whose tumors harbor the discovered cell cycle checkpoint defects and this could also benefit other patients with bladder cancer and tumor suppressor or DNA repair defects.

In one aspect, the present invention provides a method of treating urinary tract cancer in a subject in need thereof, comprising determining if there are loss-of-function mutations in the CDKN1A and RB1 genes in a biological sample from the subject; and treating the subject with a combination of a checkpoint kinase inhibitor and a DNA damaging agent if there are loss-of-function mutations in the CDKN1A and RB1 genes. In some embodiments, the urinary tract cancer is bladder cancer, and in further embodiments the subject has been diagnosed with bladder cancer

In some embodiments, the method comprises the step of obtaining a biological sample from the subject. In further embodiments, the biological sample is a bladder tissue sample.

In some embodiments, the subject is characterized as having wild-typeTP53. In additional embodiments, at least one of the loss-of-function mutations is a truncating mutation, in further embodiments the subject is also identified as having an increased level of APOBEC mutations, and in yet further embodiments the loss-of-function mutations in the CDKN1A and RB1 genes are identified using polymerase chain reaction.

In some embodiments, the checkpoint kinase inhibitor is selected from the list of Chk1 and Chk2 inhibitors consisting of bisarylurea, dibenzoazeipinone, squaric acid derivatives, furanyl indazole, benzimidazole, quinolinone, thienopyridine, and imidazopyrizine compounds, while in further embodiments the DNA damaging agent is selected from the list of agents consisting of cisplatin, cyclophosphamide, 5-fluorouracil, etoposide, or bleomycin.

A further aspect of the invention provides a method of treating urinary tract cancer in a subject in need thereof, comprising determining if there is a loss-of-function mutations in a CDKN1A gene in a biological sample from the subject; determining if there is a mutation in a second gene selected from the list consisting of RAB44, TERT, MUC16, HRNR, and FLG; and selecting specific anticancer treatment for the subject based on the identification of a mutation in the second gene. In some embodiments, the urinary tract cancer is bladder cancer.

In some embodiments, the method comprises the step of obtaining a biological sample from the subject, while in further embodiments the biological sample is a bladder tissue sample. In yet further embodiments, the mutations in the CDKN1A and second genes are identified using polymerase chain reaction.

In some embodiments, the second gene is RAB44 and the specific anticancer treatment is RAB44 inhibition. In other embodiments, the second gene is TERT and the specific anticancer treatment is treatment with a combination of a checkpoint kinase inhibitor and a DNA damaging agent. In further embodiments the second gene is MUC16 and the specific anticancer treatment is immunotherapy. In yet further embodiments the second gene is HRNR and the specific anticancer treatment is treatment with a combination of an AKT inhibitor and a DNA damaging agent.

### BRIEF DESCRIPTION OF THE FIGURES

The present invention may be more readily understood by reference to the following figures, wherein:

FIGS. 1A-1D provide graphs and schematics showing that bladder urothelial carcinomas display a high frequency of CDKN1A truncating mutations. A, CDKN1A alteration frequencies in TCGA PanCancer Atlas Studies. Alterations include mutations (white), amplifications (hashmarks), deep deletions (black), and multiple alterations (gray). B, Percentages of CDKN1A truncating mutations across the cancer types most strongly enriched for CDKN1A alterations in TCGA, in addition to lung cancers included in TCGA. C, Schematic of CDKN1A mutations. Depicted are driver truncating mutations (black, 142 total), VUS missense mutations (white, 21 total), VUS in-frame mutations (hashmarks, 2 total), and driver splice mutations (hashmarks, 1 total). D, Percentages of CDKN1A deletions that are homozygous (deep deletions, top) or heterozygous (shallow deletions, bottom).

FIGS. 2A-2C provide graphs showing that mutations in APOBEC, mismatch repair, and homologous recombination genes are enriched in tumors that also harbor a CDKN1A alteration. A, Frequencies of alteration events in genes included in the APOBEC mutational landscape in tumors that harbor a CDKN1A alteration (white) versus those that do not (black). B, Frequencies of alteration events in mismatch repair genes in tumors that harbor a CDKN1A alteration (white) versus those that do not (black). C, Frequencies of alteration events in homologous recombination genes in tumors that harbor a CDKN1A alteration (white) versus those that do not (black). A star next to the gene name indicates that the gene in statistically significantly enriched in the altered group.

FIGS. 3A-C provide graphs showing that RB1, TERT, MUC16, RAB44, HRNR, and FLG are enriched for alterations in tumors that also harbor a CDKN1A alteration. A, Frequencies of alteration events in genes with the highest frequency of alterations of any group in tumors that harbor a CDKN1A alteration (white) versus those that do not (black). B, Frequencies of alteration events in genes with the most statistically significant p vales in tumors that harbor a CDKN1A alteration (white) versus those that do not (black). C, Frequency of alteration events in FLG in tumors that harbor a CDKN1A alteration (white) versus those that do not (black). A star next to the gene name indicates that the gene in statistically significantly enriched in the altered group.

FIGS. 4A-4E provide graphs and schematics showing bladder urothelial carcinomas display a high frequency of RB1 truncating mutations. A, RB1 alteration frequencies in TCGA PanCancer Atlas Studies. Alterations include mutations (white), amplifications (hashmarks), deep deletions (black), and multiple alterations (gray). B, Percentages of RB1 truncating mutations across the cancer types most strongly enriched for RB1 alterations in TCGA, in addition to lung cancers included in TCGA. C, Venn diagram depicting tumor samples that harbored a CDKN1A alteration only (103/1425, left), an RB1 alteration only (268/1425, right), or co-occurring alterations (44/1425, center). Log 2 odds ratio 0.687, p-value=0.010, tendency for co-occurrence. D, Schematic of RB1 mutations. Depicted are driver truncating mutations (201 total), VUS missense mutations (46 total), driver missense mutations (5 total), driver splice mutations (58 total), VUS in-frame mutations (2 total), and driver SV/fusion mutations (1 total). E, Percentages of RB1 deletions that are homozygous (deep deletions, top) or heterozygous (shallow deletions, bottom).

FIG. 5 provides a flowchart showing that genomic and environmental carcinogenic mechanisms converge to promote bladder tumorigenesis. Schematic depicting two potential parallel processes that likely act in concordance to promote tumor progression in bladder urothelial carcinomas.

### DETAILED DESCRIPTION OF THE INVENTION

The present invention provides a method of treating urinary tract cancer in a subject in need thereof. The method includes determining if there are loss-of-function mutations in the CDKN1A and RB1 genes in a biological sample from the subject; and treating the subject with a combination of a checkpoint kinase inhibitor and a DNA damaging agent if there are loss-of-function mutations in the CDKN1A and RB1 genes. Alternately, the method includes determining if there is a loss-of-function mutations in a CDKN1A gene in a biological sample from the subject; determining if there is a mutation in a second gene selected from the list consisting of RAB44, TERT, MUC16, HRNR, and FLG; and selecting specific anticancer treatment for the subject based on the identification of a mutation in the second gene.

### Definitions

“Diagnosis” as used herein generally includes determination as to whether a subject is likely affected by a given disease, disorder or dysfunction. The skilled artisan often makes a diagnosis on the basis of various symptoms and/or one or more diagnostic indicators, i.e., a biomarker, the presence, absence, or amount of which is indicative of the presence or absence of the disease, disorder or dysfunction.

“Prognosis” as used herein generally refers to a prediction of the probable course and outcome of a clinical condition or disease. A prognosis of a patient is usually made by evaluating factors or symptoms of a disease that are indicative of a favorable or unfavorable course or outcome of the disease. It is understood that the term “prognosis” does not necessarily refer to the ability to predict the course or outcome of a condition with 100% accuracy. Instead, the skilled artisan will understand that the term “prognosis” refers to an increased probability that a certain course or outcome will occur; that is, that a course or outcome is more likely to occur in a patient exhibiting a given condition, when compared to those individuals not exhibiting the condition.

As used herein, the terms “treatment,” “treating,” and the like, refer to obtaining a desired pharmacologic or physiologic effect. The effect may be therapeutic in terms of a partial or complete cure for a disease or an adverse effect attributable to the disease. “Treatment,” as used herein, covers any treatment of a disease in a mammal, particularly in a human, and can include inhibiting the disease or condition, i.e., arresting its development; and relieving the disease, i.e., causing regression of the disease.

The terms “therapeutically effective” and “pharmacologically effective” are intended to qualify the amount of an agent which will achieve the goal of improvement in disease severity and the frequency of incidence over treatment of each agent by itself, while avoiding adverse side effects typically associated with alternative therapies. The effectiveness of treatment may be measured by evaluating a reduction in symptoms.

As used herein, the term “gene” refers to a nucleic acid fragment that expresses a specific protein, including regulatory sequences preceding (5′ non-coding sequences) and following (3′ non-coding sequences) the coding sequence.

The terms “mutant” and “mutation” mean any detectable change in genetic material, e.g. DNA, RNA, cDNA, or any process, mechanism, or result of such a change. This includes gene mutations, in which the structure (e.g. DNA sequence) of a gene is altered, any gene or DNA arising from any mutation process, and any expression product (e.g. protein or enzyme) expressed by a modified gene or DNA sequence. Generally a mutation is identified in a subject by comparing the sequence of a nucleic acid or polypeptide expressed by said subject with the corresponding nucleic acid or polypeptide expressed in a control population. However, a mutation in the genetic material may also be “silent”, i.e. the mutation does not result in an alteration of the amino acid sequence of the expression product.

A “biomarker” in the context of the present invention refers to a biological compound, such as a polynucleotide or polypeptide which is differentially expressed in a sample taken from a patient having bladder cancer (e.g., urine sample containing cancerous urothelial cells) as compared to a comparable sample taken from a control subject (e.g., a person with a negative diagnosis, normal or healthy subject, or subject without bladder cancer). The biomarker can be a nucleic acid, a fragment of a nucleic acid, a polynucleotide, or an oligonucleotide that can be detected and/or quantified. Bladder cancer biomarkers include polynucleotides comprising nucleotide sequences from genes or RNA transcripts of genes, including but not limited to those described herein.

The phrase “differentially expressed” refers to differences in the quantity and/or the frequency of a biomarker present in a sample taken from patients having, for example, bladder cancer as compared to a control subject or subject without cancer. For example, a biomarker can be a polynucleotide which is present at an elevated level or at a decreased level in samples of patients with bladder cancer compared to samples of control subjects. Alternatively, a biomarker can be a polynucleotide which is detected at a higher frequency or at a lower frequency in samples of patients with bladder cancer compared to samples of control subjects. A biomarker can be differentially present in terms of quantity, frequency or both.

A polynucleotide is differentially expressed between two samples if the amount of the polynucleotide in one sample is statistically significantly different from the amount of the polynucleotide in the other sample. For example, a polynucleotide is differentially expressed in two samples if it is present at least about 120%, at least about 130%, at least about 150%, at least about 180%, at least about 200%, at least about 300%, at least about 500%, at least about 700%, at least about 900%, or at least about 1000% greater than it is present in the other sample, or if it is detectable in one sample and not detectable in the other.

The terms “subject” and “patient” can be used interchangeably herein, and generally refer to a mammal, including, but not limited to, primates, including simians and humans, equines (e.g., horses), canines (e.g., dogs), felines, various domesticated livestock (e.g., ungulates, such as swine, pigs, goats, sheep, and the like), as well as domesticated pets and animals maintained in zoos. Treatment and evaluation of human subjects is of particular interest. Human subjects can be various ages, such as a child (under 18 years), adult (18 to 59 years) or elderly (60 years or older) human subject.

As used herein, the term “about” refers to +/−10% deviation from the basic value.

Unless defined otherwise, all technical and scientific terms used herein have the same meaning as commonly understood by one of ordinary skill in the art to which this invention belongs.

As used herein and in the appended claims, the singular forms “a”, “and”, and “the” include plural referents unless the context clearly dictates otherwise. Thus, for example, reference to “a sample” also includes a plurality of such samples and reference to “a biomarker” includes reference to one or more biomarkers, and so forth.

### Guiding Treatment by Detecting CDKN1 and RB1 Mutations

One aspect of the invention provides a method of treating urinary tract cancer in a subject in need thereof, comprising determining if there are mutations in the CDKN1A (cyclin dependent kinase inhibitor 1A) and RB1 (Retinoblastoma-associated protein 1) genes in a biological sample from the subject; and treating the subject with a combination of a checkpoint kinase inhibitor and a DNA damaging agent if there are mutations in the CDKN1A and RB1 genes. In some embodiments, the mutations are loss-of-function mutations.

The specific mutation in any of the urinary tract cancer-associated genes described herein can be any type of mutation in the indicated gene. Preferably the mutation is a loss-of-function mutation. A loss of function mutation is a mutation in which the altered gene product lacks the molecular function of the wild-type gene. In some embodiments, the specific mutation can occur anywhere throughout the entire coding region, resulting in a loss of function, a partial loss of function, no loss in function, or an unknown effect on function (variant of uncertain significance). In some embodiments, the mutation is a missense mutation, a frameshifting mutation, a splice-site mutation, a nonsense mutation, a complex mutation, or a silent (synonymous) mutation. The specific mutation can be a driver mutation (i.e., causative of the cancer) or can be a passenger mutation (i.e., although not causative of the cancer, it is a biomarker for the cancer).

In some embodiments, at least one of the loss-of-function mutations is a truncating mutation. Truncating mutations are mutations predicted to shorten the coding sequence of a gene. An example of a truncating mutation is a premature stop codon, which produces a truncated, usually non-functioning protein.

### Urinary Tract Cancer

“Urinary tract cancer,” as used herein, refers to any malignant disease of the urinary tract including but not limited to, adenocarcinoma, transitional cell carcinoma, squamous cell carcinoma, carcinoma in situ, clear carcinoma, granular cell carcinoma and sarcomatoid carcinoma. Urinary tract cancer is cancer of any area of the urinary tract, including the urothelium, kidney, ureter, bladder (also referred to as “urinary bladder”), lamina propria, bladder muscle and urethra. Urinary tract cancer is upper tract urothelial carcinoma (UTUC), and the bladder cancer is non-muscle invasive bladder cancer (NMIBC), MIBC, or metastatic bladder cancer. In some embodiments, the urinary tract cancer is bladder cancer.

The terms “tumor,” “cancer” and “neoplasia” are used interchangeably and refer to a cell or population of cells whose growth, proliferation or survival is greater than growth, proliferation or survival of a normal counterpart cell, e.g., a cell proliferative, hyperproliferative or differentiative disorder. Typically, the growth is uncontrolled. The term “malignancy” refers to invasion of nearby tissue. The term “metastasis” or a secondary, recurring or recurrent tumor, cancer or neoplasia refers to spread or dissemination of a tumor, cancer or neoplasia to other sites, locations or regions within the subject, in which the sites, locations or regions are distinct from the primary tumor or cancer. Neoplasia, tumors and cancers include benign, malignant, metastatic and non-metastatic types, and include any stage (I, II, III, IV or V) or grade (G1, G2, G3, etc.) of neoplasia, tumor, or cancer, or a neoplasia, tumor, cancer or metastasis that is progressing, worsening, stabilized or in remission. In particular, the terms “tumor,” “cancer” and “neoplasia” include carcinomas, such as squamous cell carcinoma, adenocarcinoma, adenosquamous carcinoma, anaplastic carcinoma, large cell carcinoma, and small cell carcinoma.

In some embodiments, the subject has been diagnosed with bladder cancer. Symptoms include blood in the urine, pain with urination, and low back pain. A preferred method for diagnosing of the state of the bladder is by way of cystoscopy, which is a procedure in which a flexible or rigid tube (called a cystoscope) bearing a camera and various instruments are inserted into the bladder through the urethra. This procedure allows for samples of suspicious lesions to be taken for a biopsy. In addition, an MRI and/or CT scan can be used to stage bladder cancer if it has been detected.

The methods described herein for treatment of urinary tract cancer may be used in individuals who have not yet been diagnosed (for example, preventative screening), or who have been diagnosed, or who are suspected of having bladder cancer (e.g., display one or more characteristic symptoms), or who are at risk of developing bladder cancer (e.g., have a genetic predisposition or presence of one or more developmental, environmental, occupational, or behavioral risk factors). In particular, a subject may be at risk of having bladder cancer because of smoking, chronic catheterization, family history, or an environmental exposure to a carcinogen. Subjects in certain occupations, such as, but not limited to, veterans, firefighters, chemists, bus drivers, rubber workers, mechanics, leather workers, blacksmiths, machine setters, or hairdressers may also be at higher risk of developing bladder cancer and benefit from diagnostic screening for bladder cancer by the methods described herein. In some embodiments, the subject being treated has an increased risk of developing urinary tract cancer as a result of being exposed to higher-than-normal levels of urinary tract carcinogens.

### Biological Samples

As used herein, a “biological sample” refers to a sample of tissue, cells, or fluid isolated from a subject, including but not limited to, for example, urine, urothelial cells, a bladder cancer biopsy, blood, buffy coat, plasma, serum, blood cells (e.g., peripheral blood mononucleated cells (PBMCS), band cells, neutrophils, monocytes, or T cells), fecal matter, bone marrow, bile, spinal fluid, lymph fluid, samples of the skin, external secretions of the skin, respiratory, intestinal, and genitourinary tracts, tears, saliva, milk, organs, biopsies and also samples of in vitro cell culture constituents, including, but not limited to, conditioned media resulting from the growth of cells and tissues in culture medium, e.g., recombinant cells, and cell components. In some embodiments, the biological sample is a bladder tissue sample.

The biological sample may comprise, for example, urine, urothelial cells, or a biopsy from a bladder cancer. In particular, the biological sample may comprise cancerous cells from a bladder tumor that are exfoliated into the urine of a subject. Such cancerous cells may be isolated from samples of urine, for example, by centrifugation. In certain embodiments, blood cells, including red blood cells and white blood cells are removed from the biological sample prior to determining biomarker levels.

The biological sample may be fresh or stored. For example, bladder cancer tissue samples may be or have been stored or banked under suitable tissue storage conditions. The urine sample may have been expressly obtained for the assays of this invention or a urine sample obtained for another purpose which can be subsampled for the assays of this invention. Preferably, urine samples are either chilled or frozen shortly after collection if they are being stored to prevent deterioration of the sample.

In some embodiments, the method further comprising the step of obtaining a biological sample from the subject. The biological sample obtained from the subject to be diagnosed is typically urine, urothelial cells, or a bladder cancer biopsy, but can be any sample from bodily fluids, tissue or cells that contain the expressed biomarkers. A “control” sample, as used herein, refers to a biological sample, such as a bodily fluid, tissue, or cells that are not diseased. That is, a control sample is obtained from a normal or healthy subject (e.g. an individual known to not have bladder cancer). A biological sample can be obtained from a subject by conventional techniques. For example, urine can be spontaneously voided by a subject or collected by bladder catheterization. Urinary cells can be collected from urine by using centrifugation to sediment cells and then discarding urinary fluid. In addition, urothelial cells may be separated from blood cells (e.g. white blood cells and red blood cells) in urine by fluorescence-activated cell sorting (FACS) or magnetic-activated cell sorting (MACS), or any other cell sorting method known in the art.

In certain embodiments, the biological sample is a bladder tumor sample, including the entire tumor or a portion, piece, part, segment, or fraction of a tumor. Solid tissue samples can be obtained by surgical techniques according to methods well known in the art. A bladder cancer biopsy may be obtained by methods including, but not limited to, an aspiration biopsy, a brush biopsy, a surface biopsy, a needle biopsy, a punch biopsy, an excision biopsy, an open biopsy, an incision biopsy or an endoscopic biopsy.

Prior to analysis for the one or more mutations, it may be preferable to purify the sample. DNA extraction methods are well known to those of skill in the art. For example, the Omni™ tissue DNA purification kit contains silica-based spin-capture columns and nontoxic reagents that are designed specifically for genomic DNA extraction from tissues and cultured cells. After sample lysis the DNA is purified through spin-column capture in less than 20 minutes.

### Detection of Mutations

The detection of the one or more mutations can be carried out by conventional means known in the art. In some embodiments, a single mutation is identified, while in further embodiments a plurality of mutations are identified. In some embodiments, the presence of the one or more mutations in the one or more urinary tract cancer-associated genes is detected by procedures such as, for example, nucleic acid sequencing, in situ hybridization, and immunohistochemistry, any of which may also involve nucleic acid amplification. Representative examples of nucleic acid sequencing techniques include, but are not limited to, chain terminator (Sanger) sequencing, dye terminator sequencing, sequencing by synthesis, pyrosequencing, and next-generation sequencing. Procedures for nucleic acid hybridization include using labeled primers or probes directed against one or more urothelial cancer-associated genes, and fixed cell preparations (fluorescence in situ hybridization). In some methods, a target urothelial cancer-associated gene may be amplified prior to or simultaneous with detection. Representative examples of nucleic acid amplification procedures include, but are not limited to, polymerase chain reaction (PCR), ligase chain reaction (LCR), strand displacement amplification (SDA), and nucleic acid sequence based amplification (NASBA). Procedures for detecting mutations in one or more urothelial cancer-associated genes include, for example, Southern blot hybridization, in situ hybridization, and fluorescence in situ hybridization (FISH). For examples of methods of detecting mutations (e.g., TERT promoter mutations) in urine samples, see US 20210130908, the disclosure of which is incorporated herein by reference. In some embodiments, the loss-of-function mutations in the CDKN1A and RB1 genes are identified using polymerase chain reaction.

In some embodiments, the methods further comprise comparing the one or more mutations in the urinary tract cancer-associated genes in the biological sample to germline mutations to filter out single nucleotide polymorphisms (SNPs), sequencing errors, and/or rare variants.

In some embodiments, the subject is characterized as having wild-type tumor protein P53 (TP53). TP53 is a tumor suppressor gene that plays a role in preventing genome mutation. Wild-type refers to a strain, gene, or characteristic which prevails among individuals in natural conditions, as distinct from an atypical mutant type. The inventors observed that in tumors with a CDKN1A alteration, 60.39% were wildtype for TP53 while 39.61% had a TP53 alteration. Therefore, patients with the combination of a CDKN1A alteration and wildtype TP53 are the molecular subgroup likely to have enhanced responsiveness to DNA damaging agents such as cisplatin.

In some embodiments, the subject is also identified as having an increased level of APOBEC mutations. APOBEC (“apolipoprotein B mRNA editing enzyme, catalytic polypeptide”) is a family of evolutionarily conserved cytidine deaminases involved in mRNA editing. The inventors found an enrichment for alteration events in APOBEC genes (e.g., PIK3CA, BRCA2, KMT2C, and ARID1A) in tumors that also harbored a CDKN1A alteration.

### Checkpoint Kinase Inhibitors and DNA Damaging Agents

The methods described herein may be used to determine how to provide improved treatment of a patient who may have urinary tract cancer. In some embodiments, the method includes treating the subject with a combination of a checkpoint kinase inhibitor and a DNA damaging agent if mutations in the CDKN1A and RB1 genes are identified. Methods of treatment using combined DNA damaging agents and checkpoint kinase inhibitors are known to those skilled in the art. Smith et al., Cancer J., 27(6):501-505 (2021). Preferably, the combination of a checkpoint kinase inhibitor and a DNA damaging agent exhibits synergistic effects for treatment of urinary tract cancer. Checkpoint kinase inhibitors include CHK1 inhibitors and CHK2 inhibitors. In some embodiments, the checkpoint kinase inhibitor is selected from the list of Chk1 and Chk2 inhibitors consisting of bisarylurea, dibenzoazeipinone, squaric acid derivatives, furanyl indazole, benzimidazole, quinolinone, thienopyridine, and imidazopyrizine compounds.

In some embodiments, a CHK1 inhibitor is used. CHK1 is a phosphorylation target of the ATR kinase and is a downstream mediator of ATR activity. Phosphorylation of CHK1 by ATR activates CHK1 activity, which in turn phosphorylates Cdc25A and Cdc25C, mediating ATR dependent DNA repair mechanisms (Wagner and Kaufmann, Pharmaceuticals 3:1311-34 (2010)).

A variety of CHK1 inhibitors are known in the art, including some that are currently in clinical trials for cancer treatment. Any known CHK1 inhibitor may be utilized in combination with a DNA damaging agent, including but not limited to XL9844 (Exelixis, Inc.), UCN-01, CHIR-124, AZD7762 (AstraZeneca), AZD1775 (Astrazeneca), XL844, LY2603618 (Eli Lilly), LY2606368 (prexasertib, Eli Lilly), GDC-0425 (Genentech), PD-321852, PF-477736 (Pfizer), CBP501, CCT-244747 (Sareum), CEP-3891 (Cephalon), SAR-020106 (Sareum), Arry-575 (Array), SRA737 (Sareum), V158411 and SCH 900776 (aka MK-8776, Merck). [See Wagner and Kaufmann, Pharmaceuticals 3:1311-34 (2010); Thompson and Eastman, Br J Clin Pharmacol 76:3 (2013); Ronco et al., Med Chem Commun 8:295-319 (2017)] CCT244747 showed anti-tumor activity in combination with gemcitabine and irinotecan, and clinical trials have been performed with LY2603618 and SCH900776 (Ronco et al., Med Chem Commun 8:295-319 (2017)).

In some embodiments, a CHK2 inhibitor is used. Examples of CHK2 inhibitors include, but are not limited to, NSC205171, PV1019, CI2, CI3 (Gokare et al., Oncotarget 7:29520-30 (2016)), 2-arylbenzimidazole (ABI, Johnson & Johnson), NSC109555, VRX0466617 and CCT241533 (Ronco et al., Med Chem Commun 8:295-319 (2017)). PV1019 showed enhanced activity in combination with topotecan or camptothecin (Ronco et al., ibid).

The method also includes the use of DNA damaging agents. Examples of DNA damage-inducing agents include CDK12 inhibitors and PARP inhibitors. See Choi W., Lee ES., Int J Mol Sci., 23(3):1701 (2022) for a discussion of the therapeutic targeting of the DNA damage response in cancer.

PARP Inhibitors are one class of DNA damaging agents. Poly-(ADP-ribose) polymerase (PARP) plays a key role in the DNA damage response and either directly or indirectly affects numerous DDR pathways, including BER, HR, NER, NHEJ and MMR (Gavande et al., 2016, Pharmacol Ther 160:65-83). A number of PARP inhibitors are known in the art, such as olaparib, talazoparib (BMN-673), rucaparib, veliparib, niraparib, CEP 9722, MK 4827, BGB-290 (pamiparib), ABT-888, AG014699, BSI-201, CEP-8983, E7016 and 3-aminobenzamide (see, e.g., Rouleau et al., Nat Rev Cancer 10:293-301 (2010). PARP inhibitors are known to exhibit synthetic lethality, for example in tumors with mutations in BRCA1/2. Olaparib has received FDA approval for treatment of ovarian cancer patients with mutations in BRCA1 or BRCA2. In addition to olaparib, other FDA-approved PARP inhibitors for ovarian cancer include nirapirib and rucaparib. Talazoparib was recently approved for treatment of breast cancer with germline BRCA mutations and is in phase III trials for hematological malignancies and solid tumors and has reported efficacy in SCLC, ovarian, breast, and prostate cancers (Bitler et al., Gynecol Oncol 147:695-704 (2017)). Veliparib is in phase III trials for advanced ovarian cancer, TNBC and NSCLC (see Wikipedia under “PARP_inhibitor”). Not all PARP inhibitors are dependent on BRCA mutation status and niraparib has been approved for maintenance therapy of recurrent platinum sensitive ovarian, fallopian tube or primary peritoneal cancer, independent of BRCA status.

CDK12 Inhibitors are another class of DNA damaging agents. Cyclin-dependent kinase 12 (CDK12) is a cell cycle regulator that has been reported to act in concert with PARP inhibitors and knockdown of CDK12 activity was observed to promote sensitivity to olaparib (Bitler et al., Gynecol Oncol 147:695-704 (2017)). CDK12 appears to act at least in part by regulating expression of DDR genes (Krajewska et al., Nature Commun 10:1757 (2019)). Various inhibitors of CDK12 are known, such as dinaciclib, flavopiridol, roscovitine, THZ1 or THZ531 (Bitler et al., Gynecol Oncol 147:695-704 (2017); Krajewska et al., Nature Commun 10:1757 (2019); Paculova & Kohoutek, Cell Div 12:7 (2017)). Combination therapy with PARP inhibitors and dinaciclib reverses resistance to PARP inhibitors.

A wide variety of other DNA damaging agents are known to those skilled in the art. In some embodiments, the DNA damaging agent is selected from the list of agents consisting of cisplatin, cyclophosphamide, 5-fluorouracil, etoposide, or bleomycin.

### Guiding Treatment by Detecting CDKN1 and a Second Gene

In another aspect, the present invention provides a method of treating urinary tract cancer in a subject in need thereof. The method includes determining if there is a mutation in a CDKN1A gene in a biological sample from the subject; determining if there is a mutation in a second gene selected from the list consisting of RAB44, TERT, MUC16, HRNR, and FLG; and selecting specific anticancer treatment for the subject based on the identification of a mutation in the second gene. In some embodiments, the mutation of the CDKN1A gene is a loss-of-function mutation.

This method includes many of the features of the previously described invention. In some embodiments, the urinary tract cancer being treated is bladder cancer. In additional embodiments, the method includes the step of obtaining a biological sample from the subject. In further embodiments, the biological sample is a bladder tissue sample. In yet further embodiments, the mutations in the CDKN1A and second genes are identified using polymerase chain reaction.

In some embodiments, the second gene is RAB44 and the specific anticancer treatment is RAB44 inhibition. Rab GTPases are master regulators of intracellular membrane trafficking and are divided into two groups. Members of the first group, consisting of Rab 1-43, are typical “small” Rab GTPases, with molecular weights of approximately 20-30 kDa. The second group consists of Rab44, Rab45 [alias RASEF (RAS and EF-hand domain-containing protein)], and CRACR2A (calcium release-activated channel regulator 2A) [alias Rab46]. Knockdown of Rab44 enhances osteoclast differentiation, and conversely, overexpression of Rab44 inhibits osteoclast differentiation. Yamguchi et al., Cell Mol Life Sci 75, 33-48 (2018). It has also been shown that that Rab44 regulates IgE-mediated anaphylaxis in mice and granule exocytosis in mast cells. Kadowaki et al., Cell Mol Immunol 17, 1287-1289 (2020). Two isoforms of Rab44 have been identified. Kadowaki et al., FEBS Open Bio., 11(4): 1165-1185 (2021).

A variety of methods are available to inhibit RAB44. Such compounds, agents, moieties, or substances can include, but are not limited to, small organic molecules, antisense nucleic acids, siRNA DNA aptamers, peptides, antibodies, non-antibody proteins, cytokines, chemokines, and chemo-attractants.

The RAB44 inhibitor can include any polynucleotide by which the expression of a target gene (e.g., RAB44) is selectively inhibited. Using RNA interference (RNAi), for example, a mediator of sequence-specific mRNA degradation (e.g., a 19 to 23-nucleotide small interfering RNA) can be produced from a longer dsRNA by digestion with ribonuclease III. A cytoplasmic RISC (RNA-induced silencing complex) binds to an siRNA and directs degradation of an mRNA comprising a sequence complementary to one strand of the siRNA. The application of RNA interference in mammals has a therapeutic gene silencing effect.

In some instances, anti-sense oligonucleotides, including anti-sense RNA molecules, and anti-sense DNA molecules, can be used that act to directly block the translation of RAB44 mRNA by binding thereto and thus preventing protein translation or increasing mRNA degradation, thus decreasing the level of Rab GTPases, and thus activity, in a cell. For example, antisense oligonucleotides of at least about 15 bases and complementary to unique regions of the mRNA transcript sequence encoding for RAB44 may be synthesized, e.g., by conventional phosphodiester techniques. Methods for using antisense techniques for specifically inhibiting gene expression of genes whose sequence is known are well known in the art (e.g., see U.S. Pat. Nos. 6,566,135; 6,566,131; 6,365,354; 6,410,323; 6,107,091; 6,046,321; and 5,981,732).

In some instances, small inhibitory RNAs (siRNAs) can also function as inhibitors of expression of RAB44 for use in the present application. RAB44 gene expression can be reduced by contacting a cell with a small double-stranded RNA (dsRNA), or a vector or construct causing the production of a small double stranded RNA, such that expression of RAB44 is specifically inhibited (i.e., RNA interference or RNAi). Methods for selecting an appropriate dsRNA or dsRNA-encoding vector are well known in the art for genes whose sequence is known (e.g., see International Patent Publication Nos. WO 01/36646, WO 99/32619, and WO 01/68836).

In some embodiments, the second gene is TERT (telomerase reverse transcriptase) and the specific anticancer treatment is treatment with a combination of a checkpoint kinase inhibitor and a DNA damaging agent. Checkpoint kinase inhibitors and DNA damaging agents have been described in detail herein. Telomerase is a ribonucleoprotein polymerase that maintains telomere ends by addition of the telomere repeat TTAGGG.

In some embodiments, the second gene is MUC16 (mucin 16) and the specific anticancer treatment is immunotherapy. MUC16 encodes a protein that is a member of the mucin family. Mucins are high molecular weight, O-glycosylated proteins that play an important role in forming a protective mucous barrier, and are found on the apical surfaces of the epithelia

Exemplary immunomodulators of use in combination therapy include anticancer antibodies such as nivolumab, pembrolizumab, atezolizumab, durvalab, ipilumumab, or avelumab, cytokines, lymphokines, monokines, a stem cell growth factor, a lymphotoxin, a hematopoietic factor, a colony stimulating factor (CSF), an interferon (IFN), parathyroid hormone, thyroxine, insulin, proinsulin, relaxin, prorelaxin, follicle stimulating hormone (FSH), thyroid stimulating hormone (TSH), luteinizing hormone (LH), hepatic growth factor, prostaglandin, fibroblast growth factor, prolactin, placental lactogen, OB protein, a transforming growth factor (TGF), TGF-α, TGF-β, insulin-like growth factor (ILGF), erythropoietin, thrombopoietin, tumor necrosis factor (TNF), TNF-α, TNF-β, a mullerian-inhibiting substance, mouse gonadotropin-associated peptide, inhibin, activin, vascular endothelial growth factor, integrin, interleukin (IL), granulocyte-colony stimulating factor (G-CSF), granulocyte macrophage-colony stimulating factor (GM-CSF), interferon-α, interferon-β, interferon-γ, interferon-λ S1 factor, IL-1, IL-1cc, IL-2, IL-3, IL-4, IL-5, IL-6, IL-7, IL-8, IL-9, IL-10, IL-11, IL-12, IL-13, IL-14, IL-15, IL-16, IL-17, IL-18 IL-21 and IL-25, LIF, kit-ligand, FLT-3, angiostatin, thrombospondin, endostatin, lymphotoxin, and the like.

In some embodiments, the second gene is HRNR (Hornerin) and the specific anticancer treatment is treatment with a combination of an AKT inhibitor and a DNA damaging agent. HRNR is an epidermal protein expressed in psoriatic lesions and in human skin during wound healing.

AKT is a downstream mediator of PI3K activity. AKT is composed of three isoforms in mammals—AKT1, AKT2 and AKT3 (Guo et al., J Genet Genomics 42:343-53 (2015)). The different isoforms have different functions. AKT1 appears to regulate tumor initiation, while AKT2 primarily promotes tumor metastasis. Following activation by PI3K, AKT phosphorylates a number of downstream effectors that have widespread effects on cell survival, growth, metabolism, tumorigenesis and metastasis. AKT inhibitors include MK2206, GDC0068 (ipatasertib), AZD5663, ARQ092, BAY1125976, TAS-117, AZD5363, GSK2141795 (uprosertib), GSK690693, GSK2110183 (afuresertib), CCT128930, A-674563, A-443654, AT867, AT13148, triciribine and MSC2363318A (Guo et al., ibid; Xing et al., Breast Cancer Res 21:78 (2019); Nitulescu et al., Int J Oncol 48:869-85 (2016)). Any such known AKT inhibitor may be used in combination therapy with anti-Trop-2 ADCs and/or DDR inhibitors. MK-2206 monotherapy showed limited clinical activity in patients with advanced breast cancer who showed mutations in PIK3CA, AKT1 or PTEN and/or PTEN loss.

In alternative embodiments, the combination therapy may be used as alone or in combination with other methods of cancer treatment such as surgery, radiation therapy, chemotherapy, immunotherapy, radioimmunotherapy, immunomodulators, vaccines, or other standard urinary tract cancer treatments. For example, bladder cancer may be treated by surgical removal of at least a portion of the bladder cancer by transurethral resection or cystectomy.

### Formulation and Administration

The present invention provides a method for treating urinary tract cancer that may include administering one or more anti-cancer compounds in a pharmaceutical composition. Examples of pharmaceutical compositions include those for oral, intravenous, intramuscular, subcutaneous, or intraperitoneal administration, or any other route known to those skilled in the art, and generally involves providing an anti-cancer compound formulated together with a pharmaceutically acceptable carrier.

When preparing the compounds described herein for oral administration, the pharmaceutical composition may be in the form of, for example, a tablet, capsule, suspension or liquid. The pharmaceutical composition is preferably made in the form of a dosage unit containing a particular amount of the active ingredient. Examples of such dosage units are capsules, tablets, powders, granules or a suspension, with conventional additives such as lactose, mannitol, corn starch or potato starch; with binders such as crystalline cellulose, cellulose derivatives, acacia, corn starch or gelatins; with disintegrators such as corn starch, potato starch or sodium carboxymethyl-cellulose; and with lubricants such as talc or magnesium stearate. The active ingredient may also be administered by injection as a composition wherein, for example, saline, dextrose or water may be used as a suitable carrier.

For intravenous, intramuscular, subcutaneous, or intraperitoneal administration, the compound may be combined with a sterile aqueous solution which is preferably isotonic with the blood of the recipient. Such formulations may be prepared by dissolving solid active ingredient in water containing physiologically compatible substances such as sodium chloride, glycine, and the like, and having a buffered pH compatible with physiological conditions to produce an aqueous solution, and rendering said solution sterile. The formulations may be present in unit or multi-dose containers such as sealed ampoules or vials.

Formulations suitable for parenteral administration conveniently comprise a sterile aqueous preparation of the active compound which is preferably made isotonic. Preparations for injections may also be formulated by suspending or emulsifying the compounds in non-aqueous solvent, such as vegetable oil, synthetic aliphatic acid glycerides, esters of higher aliphatic acids or propylene glycol.

The dosage form and amount can be readily established by reference to known treatment or prophylactic regiments. The amount of therapeutically active compound that is administered and the dosage regimen for treating a disease condition with the compounds and/or compositions of this invention depends on a variety of factors, including the age, weight, sex, and medical condition of the subject, the severity of the disease, the route and frequency of administration, and the particular compound employed, the location of the unwanted proliferating cells, as well as the pharmacokinetic properties of the individual treated, and thus may vary widely. The dosage will generally be lower if the compounds are administered locally rather than systemically, and for prevention rather than for treatment. Such treatments may be administered as often as necessary and for the period of time judged necessary by the treating physician. One of skill in the art will appreciate that the dosage regime or therapeutically effective amount of the inhibitor to be administrated may need to be optimized for each individual. The pharmaceutical compositions may contain active ingredient in the range of about 0.1 to 2000 mg, preferably in the range of about 0.5 to 500 mg and most preferably between about 1 and 200 mg. A daily dose of about 0.01 to 100 mg/kg body weight, preferably between about 0.1 and about 50 mg/kg body weight, may be appropriate. The daily dose can be administered in one to four doses per day.

For example, the maximum tolerated dose (MTD) for anti-cancer compounds can be determined in tumor-free athymic nude mice. Agents are prepared as suspensions in sterile water containing 0.5% methylcellulose (w/v) and 0.1% Tween 80 (v/v) and administered to mice (7 animals/group) by oral gavage at doses of 0, 25, 50, 100 and 200 mg/kg once daily for 14 days. Body weights, measured twice weekly, and direct daily observations of general health and behavior will serve as primary indicators of drug tolerance. MTD is defined as the highest dose that causes no more than 10% weight loss over the 14-day treatment period.

In order that the subject matter disclosed herein may be more efficiently understood, examples are provided below. It should be understood that these examples are for illustrative purposes only and are not to be construed as limiting the claimed subject matter in any manner. Throughout these examples, molecular cloning reactions, and other standard recombinant DNA techniques, were carried out according to methods described in Maniatis et al., Molecular Cloning-A Laboratory Manual, 2nd ed., Cold Spring Harbor Press (1989), using commercially available reagents, except where otherwise noted.

### EXAMPLE

### Example 1: CDKN1A/p21WAF1, RB1, FLG, and HRNR Mutation Patterns Provide Insights into Urinary Tract Environmental Exposure Carcinogenesis and Potential Treatment Strategies

Because further work is needed to identify concordant biomarkers driving bladder cancer, we sought to analyze genes dysregulated alongside CDKN1A. Aside from demonstrating the prevalence of truncating mutations in genes such as CDKN1A, RB1, and ARID1A, we also propose novel genes that may contribute to tumorigenesis in bladder carcinomas, HRNR and FLG, which are enriched for alterations in tumors that also harbor CDKN1A mutations. Moreover, we show that a similar predominance of truncations exists in chromophobe renal cell carcinomas, suggesting that DNA damaging agents may also be a therapeutic option for patients with this disease. By further elucidating co-occurring vulnerabilities in bladder tumors with CDKN1A mutations, we propose a number of avenues to explore the efficacy of potential targeted therapies in combination with standard-of-care cisplatin to help improve patient outcomes in qualifying molecular subgroups.

### Methods

TCGA PanCancer Atlas Studies analyzed on cBioPortal included the following: Adrenocortical Carcinoma, Cholangiocarcinoma, Bladder Urothelial Carcinoma, Colorectal Adenocarcinoma, Breast Invasive Carcinoma, Brain Lower Grade Glioma, Glioblastoma Multiforme, Cervical Squamous Cell Carcinoma, Esophageal Adenocarcinoma, Stomach Adenocarcinoma, Uveal Melanoma, Head and Neck Squamous Cell Carcinoma, Kidney Renal Clear Cell Carcinoma, Kidney Chromophobe, Kidney Renal Papillary Cell Carcinoma, Liver Hepatocellular Carcinoma, Lung Adenocarcinoma, Lung Squamous Cell Carcinoma, Diffuse Large B-Cell Lymphoma, Acute Myeloid Leukemia, Ovarian Serous Cystadenocarcinoma, Pancreatic Adenocarcinoma, Mesothelioma, Prostate Adenocarcinoma, Skin Cutaneous Melanoma, Pheochromocytoma and Paraganglioma, Sarcoma, Testicular Germ Cell Tumors, Thymoma, Thyroid Carcinoma, Uterine Corpus Endometrial Carcinoma, Uterine Carcinosarcoma.

Studies Analyzed on cBioPortal Included the Following; Bladder Urothelial Carcinomas: Bladder Cancer (MSK/TCGA, 2020)

Bladder Cancer (Kim et al., Eur Urol., 67:198-201 (2015)), Bladder Cancer (Iyer et al., J. Clin. Oncol., 31:3133-3140 (2013)), Bladder Cancer (Al-Ahmadie et al., Nat Genet., 48:356-358 (2016)), Bladder Cancer (Robertson et al., Cell., 171:540-556. e525 (2017)), Bladder Urothelial Carcinoma (Guo et al., Nat Genet., 45:1459-1463 (2013)), Bladder Urothelial Carcinoma (TCGA, Firehose Legacy), Non-muscle Invasive Bladder Cancer (Pietzak et al., Eur Urol., 72:952-959 (2017)), Urothelial Carcinoma (Faltas et al., Nat Genet., 48:1490-1499 (2016)); upper tract urothelial carcinomas: Upper Tract Urothelial Cancer (Sfakianos et al., Eur Urol., 68:970-977 (2015)), Upper Tract Urothelial Carcinoma (Robinson et al., Nat Commun., 10:2977 (2019)), Upper Tract Urothelial Carcinoma (Su et al., Genome Biol., 22:7 (2021)), Upper Tract Urothelial Carcinoma (Kim et al., Nat Commun., 11:1975 (2020)), Upper Tract Urothelial Carcinoma PDX (Kim et al., Nat Commun., 11:1975 (2020)); Skin Cutaneous Melanoma (TCGA, Firehose Legacy; Skin Cutaneous Melanoma (Konieczkowski et al., Cancer Discov., 4:816-27 (2014)); kidney chromophobe: Kidney Chromophobe (TCGA, Firehose Legacy); ovarian serous cystadenocarcinoma: Ovarian Serous Cystadenocarcinoma (TCGA, Firehose Legacy); lung adenocarcinoma: Lung Adenocarcinoma (Imielinski et al., Cell, 150:1107-1120 (2012)), Lung Adenocarcinoma (Schoenfeld et al., Clin Cancer Res., 26:5701-5708 (2020)), Lung Adenocarcinoma (Rizvi et al., Science, 348:124-128 (2015)), Lung Adenocarcinoma (Chen et al., Nat Genet., 52:177-186 (2020)), Lung Adenocarcinoma (TCGA, Firehose Legacy; Lung Adenocarcinoma (Ding et al., Nature, 455:1069-1075 (2008)), Non-Small Cell Cancer (Jordan et al., Cancer Discov., 7:596-609 (2017)); lung squamous cell carcinoma: Lung Squamous Cell Carcinoma (TCGA, Firehose Legacy; sarcoma: Sarcoma (Barretina et al., Nat Genet., 42:715-721 (2010)), Sarcoma (TCGA, Firehose Legacy; uterine carcinosarcoma: Uterine Carcinosarcoma (Jones et al., Nat Commun., 5:5006 (2014)), Uterine Carcinosarcoma (TCGA, Firehose Legacy); uterine corpus endometrial carcinoma: Uterine Corpus Endometrial Carcinoma (TCGA, Firehose Legacy).

### Results

Bladder Urothelial Carcinomas Display a High Frequency of CDKN1A (p21WAF1) Truncating Mutations

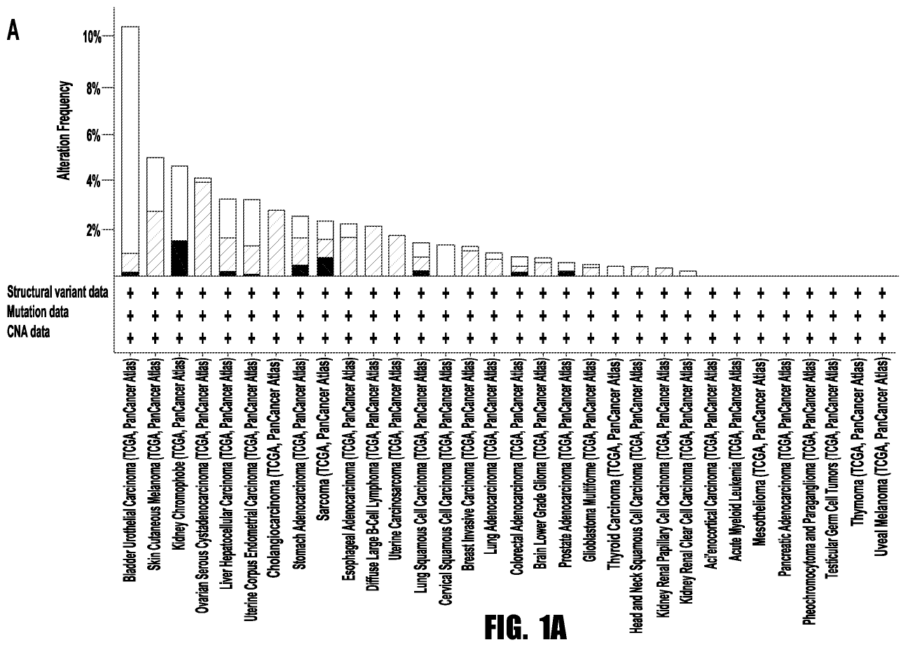

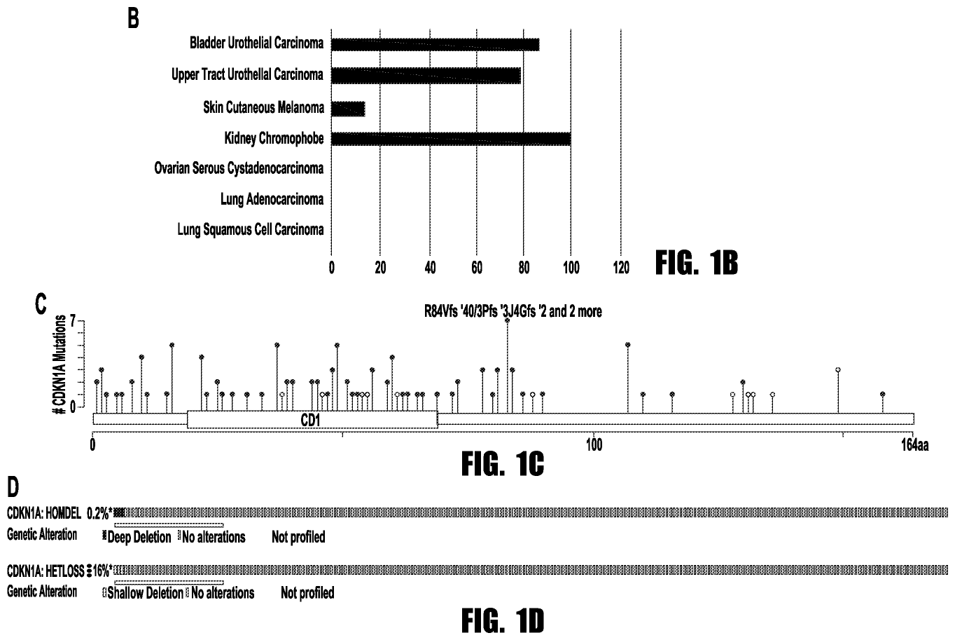

Because early work suggested that mutations in CDKN1A in cancer are rare, we asked how the alteration frequency of CDKN1A in bladder urothelial carcinomas compared to a number of other cancer types. We examined the TCGA PanCancer Atlas Studies, and found that 10.46% of all bladder urothelial carcinomas screened had a CDKN1A alteration (FIG. 1A), the highest among all cancer types included. The most common event among these alterations was mutations. While smoking is a known risk factor for the development of bladder carcinoma, only 1.44% of lung squamous cell carcinomas and 1.06% of lung adenocarcinomas displayed CDKN1A alterations, suggesting disparate driver mechanisms in bladder and lung carcinomas despite exposure to a common carcinogen.

We next sought to investigate the CDKN1A mutational landscape to better characterize particular mutations. We found that 85.54% of all bladder carcinoma CDKN1A mutations were truncating (FIG. 1B), with seven being the largest number of mutations at a single location and representing an amino acid change of R84Vfs*40/3Pfs*3/3Lfs*6/3Lfs*61/4Gfs*2 (FIG. 1C). Among the bladder carcinoma studies included in cBioPortal, the highest CDKN1A alteration frequency was 23.53%. A similar mutational pattern was also depicted in upper tract urothelial carcinomas, as 78.95% of CDKN1A mutations were truncating. We also analyzed the frequencies of particular mutations of the other cancer types most strongly enriched for CDKN1A alterations in TCGA, and, interestingly, found that 100% of mutations in kidney chromophobes were truncating. However, there was no enrichment of truncations in skin cutaneous melanoma or ovarian serous cystadenocarcinoma. Moreover, the mutational pattern seen in bladder carcinoma did not extend to that of lung cancers, despite the tobacco carcinogenesis common to both, as both lung adenocarcinomas and lung squamous cell carcinomas displayed a predominance of CDKN1A missense mutations and lacked truncating mutations entirely.

Among tumors with a CDKN1A deletion, we next asked whether there was a predominance of homozygous or heterozygous allelic loss to better understand the potential mechanisms driving sensitivity to DNA damaging agents like cisplatin. Through analysis of copy-number data for CDKN1A, we found that 0.2% of deletions were deep, representing homozygous loss, and 16% were shallow, representing heterozygous loss (FIG. 1D). This suggests that loss of a single CDKN1A allele may be sufficient to drive the subsequent phenotype of cisplatin sensitivity, with additional mechanisms in effect dependent upon mutations in other genes.

In order to better understand whether the presence of a CDKN1A mutation could serve as a biomarker for clinical prognosis, we compared the difference in overall survival for tumors with and without a CDKN1A alteration. Those that harbored a CDKN1A mutation displayed a trend toward a worse outcome with 17.97 median months of overall survival in comparison to 32.00 for those lacking a mutation, though the difference was not statistically significant. Together, these results indicate that the presence of CDKN1A inactivating truncating mutations in bladder carcinomas is likely an aberrant driver event in tumorigenesis and can also serve as a predictive biomarker for poorer clinical outcomes.

Given the significant enrichment of truncating mutations in CDKN1A, we wondered whether TP53 would also show a unique enrichment of truncations in bladder carcinomas compared to other cancer types. We found that, unlike CDKN1A, only 25.03% of TP53 mutations in bladder urothelial carcinomas were truncating, and that TP53 truncating mutations were similarly present across a number of cancer types. Moreover, in contrast to CDKN1A, the most common mutation in TP53 was missense, with 48 being the largest number of mutations at a single location and representing an amino acid change of R248Q/W/P/G. These findings suggest that there are particular genes subject to truncating mutations in bladder carcinomas, rather than a generalized pattern seen among all members of the p53 pathway. For example, other TP53 target genes, such as BBC3 and TP53I3, did not show an enrichment of truncations. When analyzing the TP53 status of tumors with a CDKN1A alteration, we found that 60.39% were wildtype for TP53 while 39.61% had a TP53 alteration. We propose that patients with the combination of a CDKN1A alteration and wildtype TP53 are the molecular subgroup likely to have enhanced responsiveness to cisplatin.

APOBEC, Mismatch Excision Repair, and Homologous Recombination Gene Mutations are Enriched in Tumors that Also Harbor a CDKN1A Mutation

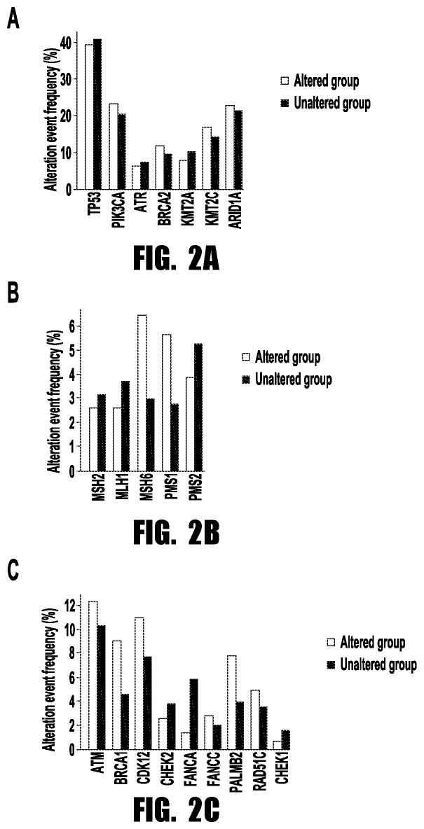

Because the APOBEC mutational signature is the predominant pattern in muscle-invasive bladder cancer, we asked whether tumors with CDKN1A alterations were enriched for any mutations in genes that are a part of this signature. We found an enrichment for alteration events in the APOBEC genes PIK3CA, BRCA2, KMT2C, and ARID1A in tumors that also harbored a CDKN1A alteration (FIG. 2A), though none of these enrichments were statistically significant. Moreover, we found statistically significant enrichments in alterations in MSH6 and PMS1, genes involved in nucleotide excision repair, in tumors that also had a CDKN1A mutation (FIG. 2B). Finally, there were statistically significant enrichments in BRCA1 and PALB2, and non-statistically significant enrichments in ATM, CDK12, FANCC, and RAD51C, genes involved in homologous recombination, in tumors that also had a CDKN1A mutation (FIG. 2C). These findings suggest that aberrant activity in a number of genes involved in DNA repair pathways may drive frameshifts that, in turn, result in downstream truncating mutations in genes like CDKN1A.

Because the APOBEC mutational signature displays a high proportion of C>T and C>G mutations, we sought to determine whether we saw a similar enrichment of this pattern in tumors with CDKN1A truncating mutations. Among bladder tumors included in cBioPortal with nonsense mutations, 54.55% harbored a C>T or C>G mutation. Moreover, among tumors with frameshift mutations, 20% of deletions were C nucleotides, and 57.89% of insertions were T or G nucleotides. These findings offer further evidence to support that CDKN1A truncations likely occur downstream of alterations in APOBEC genes.

We also aimed to further investigate the alteration events of genes included in the APOBEC mutational signature across the multiple bladder carcinoma studies in cBioPortal. Consistent with previous findings, we found that the most frequent alterations in TP53, PIK3CA, ATR, BRCA2, KMT2A, KMT2C, and ARID1A were mutations. The strongest predominance of these mutations was in muscle-invasive carcinomas, with the exception of PIK3CA and ARID1A. Interestingly, ARID1A displayed a mutational pattern similar to CDKN1A, as 66.52% of all mutations were truncating. This suggests a potential role of dysregulation of chromatin regulatory genes downstream of the APOBEC mutational landscape.

Because APOBEC genes are known to play roles in DNA repair and chromatin regulation, we also asked whether bladder carcinomas had enrichments in mutations for genes involved in mismatch excision repair or homologous recombination. We found that the most prevalent alterations in MSH2 and MSH6 were mutations. In contrast, the most predominant alterations in MLH1 and PMS2 were amplifications. Among homologous repair genes, BRCA1, PALB2, ATM, CDK12, and FANCC all showed a predominance of mutations, while RAD51C displayed a prevalence of amplifications. Together, these results suggest that an inefficiency in DNA repair in bladder carcinomas is driven by compounded mutations in APOBEC, mismatch repair, and homologous recombination genes, likely driving frameshift events downstream.

RB1, TERT, MUC16, and HRNR are the Genes with the Highest Overall Alteration Frequencies and are Enriched in Tumors that Also Harbor a CDKN1A Alteration

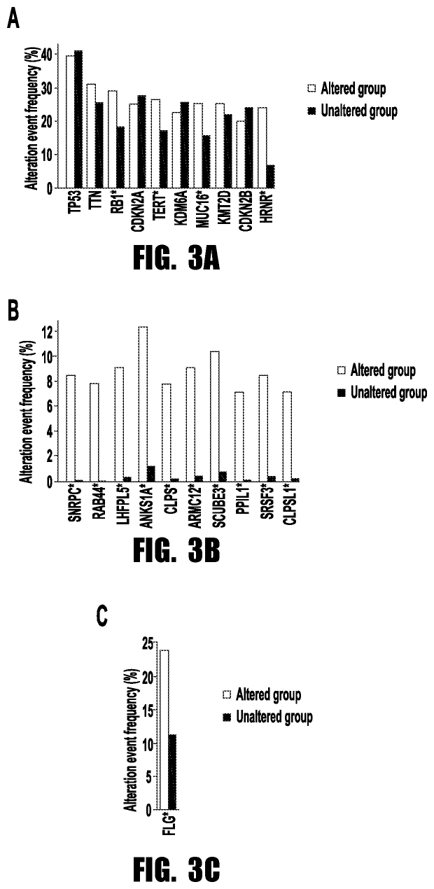

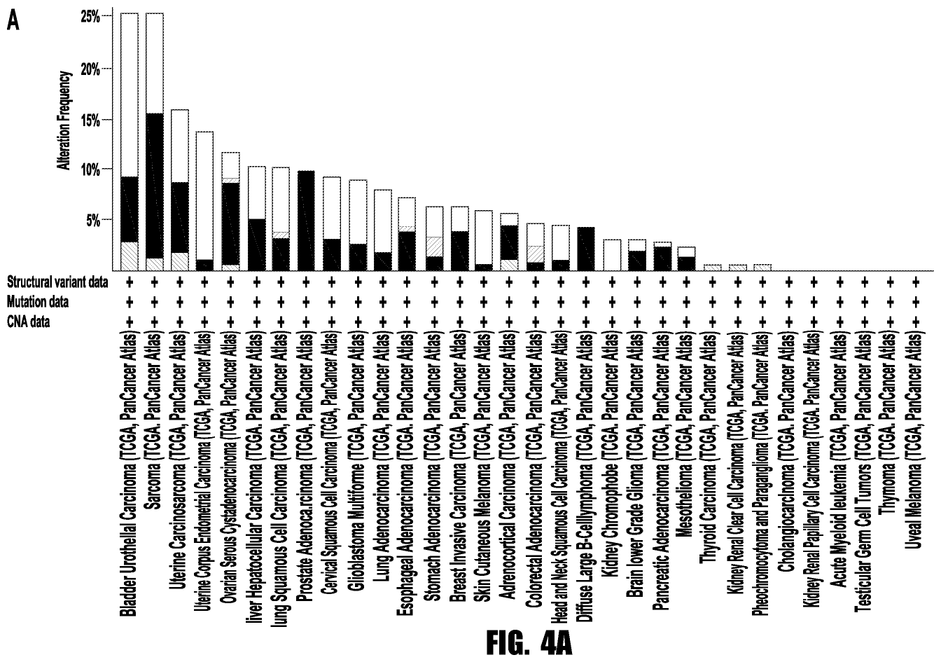

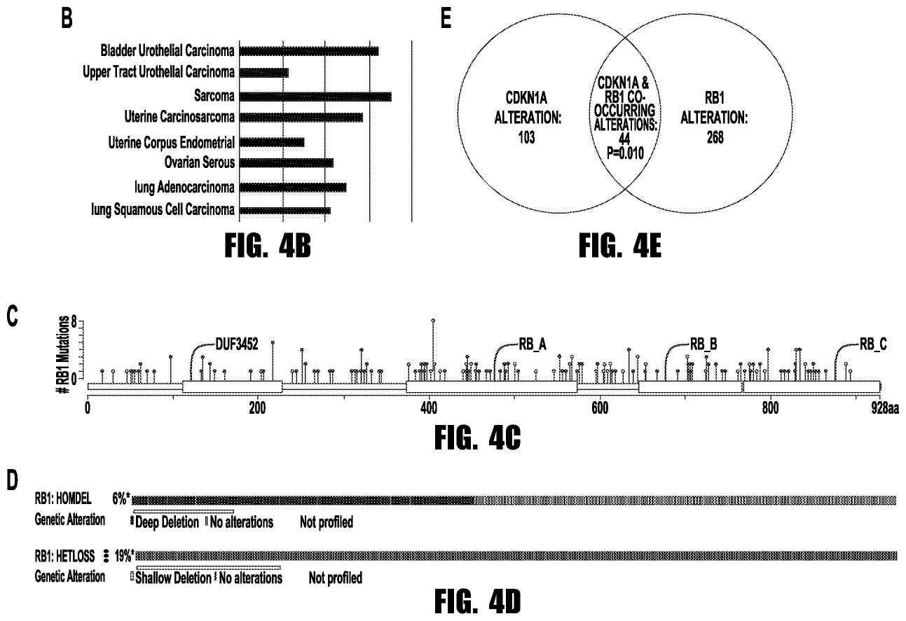

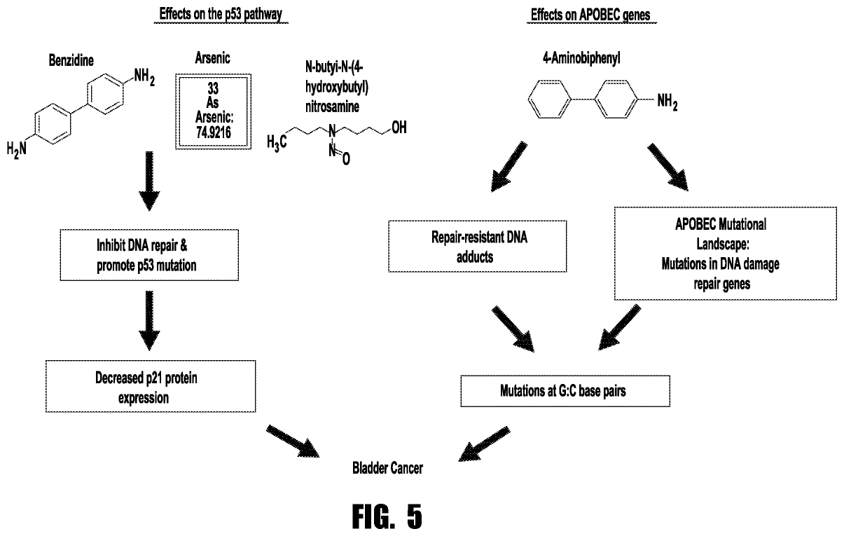

In order to nominate potential candidates for combination therapies and better understand the mechanism of action of bladder carcinogens, we first analyzed genes with the highest overall frequency of alterations that were also enriched in tumors with CDKN1A mutations. Interestingly, one such gene was RB1 (FIG. 3A), which also had the highest frequency of alterations in bladder carcinomas, 16.3%, in comparison to all other cancer types included in TCGA (FIG. 4A). Upon further analyzing RB1 alteration events, we found a predominance of mutations, with 26.47% as the highest mutation rate among all studies included in cBioPortal. Strikingly, 79.07% of these mutations were truncating (FIG. 4B), with eight being the largest number of mutations at a single location and representing an amino acid change of X405_splice (FIG. 4C). The predominance of truncating mutations in RB1 recapitulates the pattern seen in both CDKN1A and ARID1A. Among the cancer types in TCGA with the highest frequency of RB1 alterations, there was a broad enrichment of truncating mutations in RB1. RB1 truncations were particularly enriched in sarcomas and, unlike what was seen in CDKN1A, RB1 truncations were present in both lung adenocarcinomas and lung squamous cell carcinomas, suggesting a potential link between tobacco smoke and bladder tumorigenesis through dysregulation of RB1.

Similar to the analysis performed to further investigate CDKN1A deletions, we asked whether there was an enrichment of homozygous or heterozygous allelic loss among tumors with an RB1 deletion. Copy-number data for RB1 revealed that 6% of deletions were deep, indicating homozygous loss, and 19% were shallow, indicating heterozygous loss (FIG. 4D). Importantly, the predominance of heterozygous deletions in both CDKN1A and RB1 points to a potential mechanism of haploinsufficiency. Among tumors harboring a CDKN1A alteration, 30.56% also had an alteration in RB1. Most importantly from a therapeutic standpoint, among the tumors with alterations in both CDKN1A and RB1, 40.31% also had TP53 wildtype status; we propose that patients with this specific molecular profile are most likely to respond to cisplatin. Of the bladder urothelial carcinoma samples included in cBioPortal, 103 had a CDKN1A alteration, 268 had an RB1 alteration, and 44 had both CDKN1A and RB1 co-occurring alterations (FIG. 4E). Given the statistically significant tendency for CDKN1A and RB1 alterations to co-occur, this offers a promising avenue for novel targeted therapies in combination with cisplatin.

In addition to RB1, TERT, MUC16, and HRNR were among the genes with the highest overall alteration frequencies that were statistically significantly enriched in tumors also harboring a CDKN1A alteration. TERT promoter mutations and MUC16 alterations have both previously been shown to contribute to bladder tumorigenesis. Interestingly, TERT is mutated in a striking 72.38% of non-muscle invasive bladder cancers, suggesting that its role in promoting tumorigenesis may be unique to this molecular subtype. Moreover, MUC16 is mutated in as high as 38% of bladder urothelial carcinomas. On the contrary, HRNR is yet to be implicated in bladder tumorigenesis, but bladder urothelial carcinomas have the fourth highest rate of HRNR alterations across all cancer types screened in TCGA. The most frequent alterations were an equal split between mutations and amplifications. Interestingly, these findings may highlight a novel role of HRNR in driving bladder urothelial carcinomas.

RAB44 is Among the Genes with the Most Significant p-Values Enriched in Tumors that Also have a CDKN1A Alteration

We next sought to analyze the set of genes with the most significant p-values for enrichment in alterations in tumors that also have a CDKN1A mutation. One such gene among this set was RAB44 (FIG. 3B), which has previously been demonstrated to play a role in promoting bladder tumorigenesis. The predominance of alteration events in RAB44 in bladder carcinomas are amplifications, suggesting that this gene may be a viable therapeutic target.

Bladder Tumors with a CDKN1A Alteration are Enriched for Mutations in FLG

Because of the large number of environmental carcinogens that have been implicated in bladder tumorigenesis, we asked whether any genes that function in maintaining the epidermal barrier showed an enrichment of mutations in tumors that also harbor a CDKN1A mutation. One such gene, FLG, had a statistically significant enrichment for alterations in tumors with a CDKN1A mutation (FIG. 3C), and mutations were the most common event. FLG is also yet to be implicated in bladder carcinogenesis, but bladder urothelial carcinomas have the sixth highest rate of FLG alterations across all cancer types screened in TCGA. In addition to a potential novel role of FLG in promoting bladder cancer, mutations in FLG may also offer insight into a possible mechanism behind environmental exposure to carcinogens.

### Discussion

We report the prevalence of truncating mutations in both CDKN1A and RB1, with a statistically significant tendency of these alterations to co-occur. These unusual gene mutation signatures likely reflect unique pathways of carcinogen exposure through the environment with accumulation of carcinogens or their metabolites in the bladder. It has previously been demonstrated that CDKN1A mutations render cells unable to halt the cell cycle and efficiently repair DNA damage, leading to apoptosis. These CDKN1A truncating mutations therefore not only drive sensitivity to cisplatin (Sikder et al., Mol Cancer Res., 19:403-413 (2021)), but also offer the possibility for combination therapies that additionally target RB1. RB1 knockout has been shown to enhance bladder tumorigenesis both in vitro and in vivo (Wang et al., Int J Oncol., 50:1221-1232 (2017)), and it has been demonstrated that RB-deficient tumor cells have a greater dependence on CHK1 (Witkiewicz et al., Cell Rep., 22:1185-1199 (2018)), a key regulator of the DNA damage response (DDR) which enables DNA repair and allows for cell cycle progression. Bladder tumors with deficiency of the tumor suppressor RB1 have defects in the G1 checkpoint, driving genomic instability.

Therefore, we propose that tumors with co-occurring CDKN1A and RB1 loss-of-function truncations may show enhanced sensitivity to a spectrum of precision therapies with ATR, ATM, CHK1, and CHK2 inhibitors. Preclinical studies have demonstrated that CHK1 inhibitors in combination with cisplatin (Li et al., Oncotarget., 7:1947-1959 (2016)) or gemcitabine (Isono et al., Sci Rep., 11:10181 (2021)) potentiate the anticancer activity of these chemotherapeutic drugs. Inhibition of the DDR drives checkpoint abrogation, inhibition of DNA repair, and induction of cell death. Additional work remains to be done in order to determine how these combination therapies enhance the efficacy of cisplatin in patients with CDKN1A alterations, RB1 alterations, or co-occurring alterations, and whether these treatments could be viable therapeutic options in the clinic for patients with qualifying genomic alterations.

Further investigation is needed to unravel the molecular pathways by which environmental carcinogens cause bladder cancer at high rates, such as in Rhode Island and other New England States, as well as to establish strategies for prevention. Our findings warrant further experimentation to determine whether the combination of checkpoint kinase inhibitors with cisplatin will offer more efficacious personalized therapeutics for patients with tumors that harbor cell cycle checkpoint defects.

### Relationships Between APOBEC and Truncating Mutational Patterns in CDKN1A and RB1 in Bladder Carcinomas

Of the bladder tumors in TCGA, 80% display the APOBEC mutational signature (Glaser et al., Oncotarget., 9:4537-4548 (2017)). The enrichment for mutations in genes that are part of the APOBEC mutational profile in tumors that also harbor CDKN1A mutations further compounds an inefficiency in DNA repair, and, importantly, offers a number of targets for precision therapies.

Mutations in the APOBEC gene BRCA2 have been correlated with heritable risks for urothelial carcinomas, as it has been demonstrated that there are significantly higher rates of germline pathogenic variants in BRCA2 compared to cancer-free controls (Nassar et al., Genet Med., 22:709-718 (2020)). Interestingly, a rare variant in BRCA2 has been associated with an increased risk of developing both urinary tract and lung cancers (Ge et al., Sci Rep., 6:33542 (2016)). Here, we show that tumors with CDKN1A mutations are enriched for mutations in BRCA1 and BRCA2, both of which contribute to DNA repair and transcriptional regulation in response to DNA damage (Yoshida and Miki, Cancer Sci., 95:866-871 (2004)). This suggests that combination therapy with PARP inhibitors, which preferentially kill BRCA-mutated cancer cells (Chen, Chin J Cancer. 2011; 30:463-471 (2011)), may benefit a subgroup of patients with this particular mutational landscape.

APOBEC activity has also been identified as a key driver of PIK3CA mutagenesis, a gene which we demonstrate to be preferentially enriched for alterations in tumors with CDKN1A mutations. Activating mutations in PIK3CA, which encodes the catalytic subunit of phosphatidylinositol 3-kinase involved in the PI3K/AKT signaling pathway, are common oncogenic drivers of bladder carcinogenesis. This suggests that patients with PIK3CA mutations may benefit from PI3K-targeted therapies, including PI3K, mTOR, and AKT inhibitors (Henderson et al., Cell Rep., 7:1833-1841 (2014)), in combination with cisplatin-based therapy.

ARID1A is another gene that is part of the APOBEC mutational landscape, which we show to also be preferentially enriched for mutations in tumors that harbor CDKN1A alterations. ARID1A is a subunit of the SWI/SNF complex, which plays a role in ATP-dependent chromatin remodeling, thereby influencing transcriptional accessibility and modulating DNA repair (Ferguson et al., bioRxiv. 2021.2001.2012.426383 (2021)). In tumors with ARID1A mutations, EZH2 inhibition is synthetic lethal, suppressing cell growth and promoting apoptosis (Alldredge and Eskander, Gynecol Oncol Res Pract., 4:17 (2017)). Bladder tumors with ARID1A deficiencies have previously been shown to be sensitive to the small molecule EZH2 inhibitor GSK-126. Here, we demonstrate that ARID1A mutations in bladder carcinomas are predominantly inactivating truncating mutations, helping to explain sensitivity to EZH2 inhibitors and offering support for the combination of cisplatin and EZH2 inhibitors. Moreover, because mutations in ARID1A have been shown to confer sensitivity to pan-HDAC inhibitors (Fukumoto et al., Cell Rep., 22:3393-3400 (2018)), our findings offer additional evidence for the repurposing of pan-HDAC inhibitors for patients whose mutational profiles fall within this subgroup.

RAB44, TERT, MUC16, HRNR, and FLG Mutations are Enriched in Bladder Cancers with CDKN1A Alterations

The enrichment of mutations in a number of genes, including those that we propose to have novel roles in promoting bladder cancer, in tumors that also harbor a CDKN1A mutation offers new options for therapeutic intervention. First, the Ras oncogene related protein RAB44, a Rab GTPase, has previously been shown to form an oncogenic fusion protein with CDKN1A (Sun et al., bioRxiv, 111856 (2019)). Because RAB44 is not expressed in all normal tissue types and the fusion protein has a relatively high prevalence in bladder cancer, this specificity suggests that therapies targeting RAB44 may be a clinical option for patients with these fusion events.

Additionally, TERT, an important element of telomerase expression, was highly enriched in tumors with a CDKN1A alteration. TERT promoter mutations are the most common somatic lesion in bladder cancer and have been demonstrated to be a predictor of both poor survival and disease recurrence (Rachakonda et al., Proc Natl Acad Sci USA., 110:17426-31 (2013)). The resulting increased expression of telomerase downstream of TERT promoter mutations offers an attractive target for therapeutic intervention. Therefore, tumors that harbor a CDKN1A alteration may be particularly sensitive to combination therapies with cisplatin and small molecule inhibitors targeting telomere- and telomerase-associated proteins.

Moreover, MUC16, a type of Type 1 transmembrane mucin, was enriched in tumors that also have a CDKN1A alteration. MUC16 has been shown to play a role in angiogenesis as well as mediating metastasis in advanced bladder cancer (Suh et al., Chemotherapy: Open Access. 2017:06). As a result, MUC16 alteration in the presence of CDKN1A alteration may serve as a predictive biomarker for clinical prognosis. Excitingly, MUC16 mutation has been associated with an enhanced response to immune checkpoint inhibitors in patients with solid tumors (Yoon et al., Cancer Prev Res (Phila) 5:299-308 (2012)), suggesting that immunotherapies may be a viable therapeutic option for patients with co-occurring CDKN1A and MUC16 mutations.

Another gene strongly enriched in bladder tumors with CDKN1A mutations was hornerin (HRNR). Hornerin is a member of the S100 calcium-binding protein family, which is involved in the regulation of transcription factors, cell proliferation, differentiation, and death (McKiernan et al., Tumor Biol., 32:441-450 (2011)). Though HRNR is yet to be implicated in bladder carcinogenesis, its overexpression has been demonstrated in hepatocellular carcinoma tumor progression and is correlated with poor prognosis in HCC (Fu et al., BMC Cancer, 18:815 (2018)). HRNR is necessary to promote AKT phosphorylation, which is required for its activation, and is essential for metastatic pathways. Therefore, our findings suggest that AKT inhibitors may be a potential therapeutic option in combination with cisplatin for patients that harbor HRNR mutations.

From a mechanistic perspective, we demonstrate that tumors with CDKN1A mutations are also strongly enriched for mutations in the filaggrin gene (FLG), which encodes a protein product that plays a role in both structural and physiological functions in the skin (Skaaby et al., PLOS One., 9:e99437 (2014)). Importantly, FLG plays a role in protecting the skin against the uptake of chemicals upon dermal exposure. It is likely that patients that harbor a mutation in these tumors also have a total loss-of-function mutation in the FLG gene. Importantly, dermal exposure to aromatic amines and polycyclic aromatic hydrocarbons is a known risk factor for bladder cancer (Burger et al., Eur Urol., 63:234-241 (2013)). An enrichment of FLG mutations in tumors with CDKN1A mutations suggests that these patients may also have an impaired skin barrier function. Resultantly, there is likely a subsequent enhanced absorption of chemicals (Liljedahl et al., Sci Total Environ., 653:45-54 (2019)), leading to more profound impacts on DNA and driving carcinogenesis.

Emerging Insights into Bladder Cancer Environmental Carcinogenesis from CDKN1A Mutational Patterns

Rhode Island has for years had the greatest incidence of bladder cancer in the United States for both men and women, a statistic driven both by cigarette smoking and occupational exposures (Faricy-Anderson et al., Med Health R I., 93:308, 313-6 (2010)). In the 1950s, Rhode Island exceeded the national average in cigarette smoking, and given the long latency period of bladder cancer development, this historical tobacco use may play a role in recent elevated rates. Moreover, the long history of New England in the textile industry helps to explain the occupation-related exposure to carcinogens, which also have a latency period of 20 years or more. Together, the delayed effects of these historical exposures continue to be seen currently, and Rhode Island is in particularly desperate need of a better understanding of the mechanisms by which carcinogens promote bladder malignancies.

Multiple environmental carcinogens are known to drive bladder tumorigenesis, and a number of these have direct effects on TP53, CDKN1A, and their signaling pathway (FIG. 5). A thiolated arsenic metabolite, dimethylmonothioarsinic acid, has been shown to cause a decrease in both p21 and p53 protein expression, accompanied by an increase in DNA damage and intracellular hydroxyl radicals (Naranmandura et al., Chem Res Toxicol., 24:1586-1596 (2011)). Beyond interfering with the DNA damage response, arsenic has also been shown to result in a decrease in RB1 phosphorylation (Muenyi et al., Biomolecules, 5:2184-93 (2015)), thereby also cell disrupting cycle regulation. Moreover, N-butyl-N-(4-hydroxybutyl)nitrosamine, an N-nitrosamine, is a compound that has been identified as a carcinogen specific to bladder cancer in animal studies. Upon treatment with this N-nitrosamine, there has been a demonstrated decrease in p21 protein expression (Wang et al., PLOS One., 11:e0159102 (2016)), suggesting that lower p21 expression is a potential biomarker for tumorigenesis.

Additionally, 4-Aminobiphenyl (4-ABP) is an aromatic amine generated predominantly from cigarette smoking, and its metabolites have been shown to form repair-resistant DNA adducts. It has previously been demonstrated that 82.9% of mutations induced by 4-ABP occurred at G:C base pairs (Yoon et al., Cancer Prev Res (Phila) 5:299-308 (2012)); as previously stated, a majority of APOBEC mutations are C>G, suggesting that 4-ABP may play a role in the mutagenesis of APOBEC genes. Moreover, a dose-dependent response has been demonstrated between 4-ABP and impaired DNA repair capacity (Lin et al., Cancer Epidemiol Biomarkers Prev., 14:1832-1836 (2005)). 4-ABP preferentially forms adducts at two specific codons within the TP53 gene (Feng et al., Carcinogenesis, 23:1721-1727 (2002)); interestingly, mutations at these codons rarely occur in lung cancer. This specificity of 4-ABP for unique TP53 codons can help explain the TP53 mutational spectrum seen in bladder cancer, and points to potential downstream dysregulation of CDKN1A.

The mechanism of action of benzidine, a known bladder carcinogen, may offer another route for a targeted therapy. Benzidine's structure as an aromatic amine allows it to act as an intercalating agent, likely leading to downstream frameshifts and thereby promoting carcinogenesis. Benzidine has been shown to interact with DNA through both minor groove binding and partial intercalation (Amutha et al., Chemical Physics Letters, 344:40-48 (2001)). Moreover, benzidine has been found to downregulate p21 mRNA levels as well as decrease p21 protein levels (Zhao et al., Oncol Lett., 16:4628-4634 (2018)), provoking the transition of cells from G1 to S and G2. Upon treatment with a MAPK inhibitor, the effects of benzidine on p21 were suppressed. Interestingly, exposure of normal urothelial cells to smoke, a known bladder carcinogen, has been shown to drive MAPK activation (Hayashi et al., Int J Mol Sci., 21:6072 (2020)). Together, these findings offer support for a combination therapy of cisplatin and MAPK inhibitors, which have previously been shown to induce apoptosis in bladder cancer cell lines (Kumar et al., Int J Oncol., 34:1557-1564 (2009)).

### High Frequency of CDKN1A and RB1 Truncating Mutations in Chromophobe Renal Cell Carcinomas

We demonstrate that in addition to bladder and upper tract urothelial carcinomas, kidney chromophobes are also strongly enriched for CDKN1A and RB1 truncations. Further investigation is needed to determine whether other cancer types in addition to these display a similar mutational profile. Given the proximity of the kidneys to the bladder anatomically, this indicates a potential preference of organs involved in urine processing for the enrichment of CDKN1A and RB1 truncations. Importantly, our findings suggest that patients with kidney chromophobes, particularly those with metastatic disease who currently have limited treatment options (Volpe et al., BJU Int., 110:76-83 (2012)), may benefit from cisplatin-based therapies or other DNA damaging agents.

Moreover, patients with kidney chromophobes with a CDKN1A or an RB1 alteration display a trend toward poorer survival, suggesting that dysregulation of these genes may also serve as prognostic biomarkers for this cancer type. If other cancers beyond bladder, upper tract urothelial carcinomas, and kidney chromophobe tumors do indeed display enrichments of these truncations, this could expand options for precision therapies, as these tumors are likely to also be sensitive to DNA damaging agents and, potentially, the combination therapies described above.

It has been shown that 18% of TCGA samples have a strong APOBEC mutation signature (Seplyarskiy et al., Genome Res., 26:174-182 (2016)). Therefore, additional analysis is needed to determine whether other cancer types with a predominance of this mutational landscape, such as breast, cervical, and head and neck, are also marked by the prevalence of downstream truncating mutations in CDKN1A and RB1. This would provide further insight into the potential mechanisms driving these truncations and, in turn, may help predict sensitivity to cisplatin-based therapies and other DNA-damaging agents.

### Implications of Truncating Mutations in CDKN1A and RB1 for Therapeutics of Bladder Cancer and Renal Cell Chromophobe Carcinomas

The findings reported here identify a high prevalence of inactivating truncating mutations in CDKN1A, RB1, and ARID1A, likely acting downstream of the APOBEC mutational landscape. We propose a mechanism whereby upstream mutations in APOBEC genes and other genes involved in DNA repair processes drive frameshifts and, in turn, downstream truncating mutations arise. Interestingly, 4-ABP, and potentially other environmental carcinogens, may play a role in driving mutations in APOBEC genes. In addition, a number of environmental carcinogens act on the p53 signaling pathway, resulting in decreased levels of p21 mRNA levels and protein expression downstream. The two parallel processes of dysregulation at the level of the p53 pathway and alterations among APOBEC genes likely converge to promote bladder tumorigenesis.

Due to increases in therapeutic resistance, the classification of patients into distinct molecular subgroups is needed in order to enhance responsiveness to treatment. Previous work has demonstrated that patients who exhibited a better response to neoadjuvant chemotherapy had alterations in one or more of the three DNA repair genes ATM, RB1, and FANCC (Plimack et al., Eur Urol., 68:959-967 (2015)). Here, we propose that patients with co-occurring truncating mutations in CDKN1A and RB1 who also retain wildtype TP53 status are likely to respond most favorably to cisplatin-based therapies and other DNA-damaging agents. Additionally, a prior study suggested that p53 status, as measured by mRNA expression, is a predictor of de novo and induced chemoresistance (Choi et al., Cancer Cell, 25:152-165 (2014)). Under normal circumstances, p53 will activate the cell cycle checkpoint, increasing CDKN1A expression and, in turn, this promotes DNA damage-induced apoptosis. However, in the presence of CDKN1A alterations, p53's ability to trigger the checkpoint is ineffective, driving sensitivity to cisplatin. Further work is needed to determine whether patients with both CDKN1A and RB1 alterations coupled with wildtype TP53 status have improved clinical benefit, as this could define a new genomic signature to predict chemotherapy sensitivity.

Additional investigation is required to determine whether homozygous deletions in CDKN1A and RB 1 render sensitivity to cisplatin to the same extent as heterozygous deletions. Moreover, further studies are needed to determine whether there is a co-occurrence of CDKN1A mutation and heterozygous CDKN1A allelic deletion in individual tumor samples, as this would result in biallelic loss. Because we found a predominance of heterozygous allelic loss in both CDKN1A and RB1, we propose a potential mechanism of haploinsufficiency, whereby loss of both alleles of CDKN1A is not necessary for the resultant phenotype of sensitivity to cisplatin if co-occurrence of heterozygous loss of both CKND1A and RB1 is present. Our findings offer support for the molecular testing of patients prior to receiving chemotherapy to select for those most likely to respond to treatments and to therefore increase the likelihood of survival.

Most importantly, these findings offer insights into pathways of bladder cancer carcinogenesis through unique truncating mutational signatures, and the potential for a wide range of innovative clinical therapies by targeting a number of actionable genes most frequently mutated in tumors that also harbor CDKN1A truncations. Because treatment options are limited for patients with bladder carcinomas, these findings offer support to investigate the potential of checkpoint kinase inhibitors in combination with cisplatin-based therapies both in vitro and in vivo, with hope of future translation into effective personalized clinical therapeutic options.

The complete disclosure of all patents, patent applications, and publications, and electronically available material cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A method of treating urinary tract cancer in a subject in need thereof, comprising
determining if there are loss-of-function mutations in the CDKN1A and RB1 genes in a biological sample from the subject; and treating the subject with a combination of a checkpoint kinase inhibitor and a DNA damaging agent if there are loss-of-function mutations in the CDKN1A and RB1 genes.

2. The method of claim 1, wherein the urinary tract cancer is bladder cancer.

3. The method of claim 1, wherein the subject has been diagnosed with bladder cancer

4. The method of claim 1, further comprising the step of obtaining a biological sample from the subject.

5. The method of claim 1, wherein the biological sample is a bladder tissue sample.

6. The method of claim 1, wherein the subject is characterized as having wild-typeTP53.

7. The method of claim 1, wherein at least one of the loss-of-function mutations is a truncating mutation.

8. The method of claim 1, wherein the subject is also identified as having an increased level of APOBEC mutations.

9. The method of claim 1, wherein the loss-of-function mutations in the CDKN1A and RB1 genes are identified using polymerase chain reaction.

10. The method of claim 1, wherein the checkpoint kinase inhibitor is selected from the list of Chk1 and Chk2 inhibitors consisting of bisarylurea, dibenzoazeipinone, squaric acid derivatives, furanyl indazole, benzimidazole, quinolinone, thienopyridine, and imidazopyrizine compounds.

11. The method of claim 1, wherein the DNA damaging agent is selected from the list of agents consisting of cisplatin, cyclophosphamide, 5-fluorouracil, etoposide, or bleomycin.

12. A method of treating urinary tract cancer in a subject in need thereof, comprising
determining if there is a loss-of-function mutations in a CDKN1A gene in a biological sample from the subject;
determining if there is a mutation in a second gene selected from the list consisting of RAB44, TERT, MUC16, HRNR, and FLG; and
selecting specific anticancer treatment for the subject based on the identification of a mutation in the second gene.

13. The method of claim 12, wherein the urinary tract cancer is bladder cancer.

14. The method of claim 12, further comprising the step of obtaining a biological sample from the subject.

15. The method of claim 12, wherein the biological sample is a bladder tissue sample.

16. The method of claim 12, wherein the mutations in the CDKN1A and second genes are identified using polymerase chain reaction.

17. The method of claim 12, wherein the second gene is RAB44 and the specific anticancer treatment is RAB44 inhibition.

18. The method of claim 12, wherein the second gene is TERT and the specific anticancer treatment is treatment with a combination of a checkpoint kinase inhibitor and a DNA damaging agent.

19. The method of claim 12, wherein the second gene is MUC16 and the specific anticancer treatment is immunotherapy.

20. The method of claim 12, wherein the second gene is HRNR and the specific anticancer treatment is treatment with a combination of an AKT inhibitor and a DNA damaging agent.
